SEQUENCE LISTING (7 sequences)

SEQ ID NO: 1 - DNA, 44 bp, Artificial Sequence
  Synthetic - DI (version linked to cholesterol)
ccctcactcacctcatcccactcctacctaaaccaaaaaaaaaa

SEQ ID NO: 2 - DNA, 39 bp, Artificial Sequence
  Synthetic - DI (version linked to antibody)
ccctcactcacctcatcccactcctacctaaaccaaaaa

SEQ ID NO: 3 - DNA, 58 bp, Artificial Sequence
  Synthetic - DM1
ggtttaggtaggagtgggatgaggccaaatcctcatcccactcctaccactcactccc

SEQ ID NO: 4 - DNA, 53 bp, Artificial Sequence
  Synthetic - DM2
aaaaacctcatcccactcctacctaaaccggtaggagtgggatgaggatttgg

SEQ ID NO: 5 - DNA, 29 bp, Artificial Sequence
  Synthetic - CSDI-Biotin
tttttggtttaggtaggagtgggatgagg

SEQ ID NO: 6 - DNA, 39 bp, Artificial Sequence
  Synthetic - TDI
tttttggtttaggtaggagtgggatgaggtgagtgaggg

SEQ ID NO: 7 - DNA, 34 bp, Artificial Sequence
  Synthetic - TDM1
gggagtgagtggtaggagtgggatgaggatttgg

(19) United States
(12) Patent Application Publication
(10) Pub. No.: US 2023/0151405 A1
(43) Pub. Date: May 18, 2023

(54) BIODEGRADABLE DNA-ALGINATE CONJUGATE FOR REVERSIBLE PROTEIN AND CELL LABELING AND IMAGING

(71) Applicant: THE PENN STATE RESEARCH FOUNDATION, University Park, PA (US)

(72) Inventors: Yong WANG, State College, PA (US); Brandon J. DAVIS, State College, PA (US); Peng SHI, State College, PA (US)

(21) Appl. No.: 17/919,225

(22) PCT Filed: Apr. 15, 2021
(86) PCT No.: PCT/US2021/027526

§ 371 Date: Oct. 14, 2022

Related U.S. Application Data
(60) Provisional application No. 63/010,146, filed on Apr. 15, 2020.

(51) Int. Cl. C12Q 1/6804; G01N 33/53; G01N 33/58

(52) U.S. Cl. CPC C12Q 1/6804; G01N 33/5308; G01N 33/582; G01N 2458/10

(57) ABSTRACT

The present invention provides methods for signal amplification. The methods use DNA hybridization chain reaction to build labeled nanoscaffolds off of target analytes. The methods are reversible, as the detectable signal can be removed using DNA hybridization and hydrolysis.

### CROSS-REFERENCE TO RELATED APPLICATIONS

This application claims priority to U.S. Application No. 63/010,146 filed on Apr. 15, 2020, the contents of which are incorporated by reference in their entireties.

### STATEMENT REGARDING FEDERALLY FUNDED RESEARCH OR DEVELOPMENT

This invention was made with government support under Grant No. CBET1802953 awarded by the National Science Foundation. The Government has certain rights in the invention.

### SEQUENCE LISTING

This invention was made with government support under Grant No. CBET1802953 awarded by the National Science Foundation. The Government has certain rights in the invention. A Sequence Listing accompanies this application and is submitted as an ASCII text file of the sequence listing named “900905 00019 ST25.txt” which is 1.75 KB in size and was created on Apr. 14, 2021. The sequence listing is electronically submitted via EFS-Web with the application and is incorporated herein by reference in its entirety.

### BACKGROUND

The ability to stain and detect cells and biomolecules (e.g., proteins and mRNA) is critical to not only basic life science studies but also various diagnostic and therapeutic modalities. Thus, great efforts have been made to develop numerous reagents and methods for biomolecular staining and imaging. For instance, a common method for cell labeling and imaging is to treat a sample with a primary antibody that can recognize a specific cell receptor. This primary antibody is either directly labeled with fluorophores for imaging or recognized by a fluorophore-labeled secondary antibody. However, one antibody can only carry a few organic fluorophores, as the antibody would lose its binding ability if it carried too many fluorophores. As a result, a target biomolecule or cell will be labeled with a limited number of fluorophores, which significantly limits the detectable signal. This limitation is especially challenging for investigating a sample with a low number of cells or with a biomolecule that is expressed at low levels.

Various signal amplification methods have been developed to enhance signal output. For instance, biotinylated secondary antibody for labelling is one of the most effective amplification techniques routinely used. In this method, biotin strongly binds to fluorescently labeled streptavidin probes, increasing the number of fluorophores for each target molecule. Similarly, the secondary antibody can be conjugated with a polymer that carries multiple fluorophores. While these methods can indeed lead to signal amplification, the signal may only be within one order of magnitude of that generated by traditional methods. Researchers have also used inorganic nanoparticles, particularly, quantum dots, to replace organic fluorophores. Each nanoparticle can exhibit fluorescence intensity that is similar to tens of organic fluorophores, resulting in a stronger signal with fewer molecules. However, nanoparticles are prone to aggregation and slow diffusion, issues that make biomolecular labeling difficult. Moreover, it is challenging to remove inorganic nanoparticles from samples. The ability to remove fluorophores after the sample is examined would allow for multiplexed labelling and imaging or for post-detection cell separation. Nucleic acid hybridization has also been studied for signal amplification to examine mRNA.

### SUMMARY OF THE DISCLOSURE

The present invention provides a system and methods for reversibly detecting a target analyte in a sample. The methods comprise: (a) contacting the sample with a probe that comprises an initiator single-stranded DNA molecule (ssDNA) conjugated to a targeting agent that binds to the target analyte; (b) washing the sample to remove unbound probe; (c) contacting the probe-target analyte complex with: (i) a first DNA hairpin comprising (1) a first portion that is complementary to both a part of the initiator ssDNA and a part of a second DNA hairpin, and (2) a second portion that is complementary to a part of a first portion of the second DNA hairpin; and (ii) the second DNA hairpin comprising a first portion that is complementary to a part of the second portion of the first DNA hairpin and a second portion that is complementary to a part of the first portion of the first DNA hairpin; wherein either the first hairpin or the second hairpin is linked to an alginate; and wherein the initiator ssDNA, the first DNA hairpin, and the second DNA hairpin undergo hybridization chain reaction (HCR) when in contact, thereby forming a nanoscaffold attached to the targeting agent; and (d) contacting the nanoscaffold of step (c) with a detectable label that binds to the alginate; and (e) detecting the detectable label.

In some embodiments, the methods further comprise: (f) removing the detectable label from the sample by contacting the sample with a depolymerization agent selected from a complementary DNA (cDNA), alginate lyase, DNase, or any combination thereof.

In some embodiments, the methods further comprise: (g) repeating steps (a)-(e) using a different targeting agent to detect a different target analyte.

In a second aspect, the present invention provides kits for detecting a target analyte in a sample. The kits comprise: (a) a probe that comprises an initiator single-stranded DNA molecule (ssDNA) conjugated to a targeting agent that binds to the target analyte; (b) a first DNA hairpin comprising (1) a first portion that is complementary to both a part of the initiator ssDNA and a part of a second DNA hairpin, and (2) a second portion that is complementary to a part of a first portion of the second DNA hairpin; (c) a second DNA hairpin comprising a first portion that is complementary to a part of the second portion of the first DNA hairpin and a second portion that is complementary to a part of the first portion of the first DNA hairpin; wherein the first DNA hairpin or second DNA hairpin is linked to alginate; and (d) a detectable label that binds to the alginate or is conjugated to the alginate.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows a characterization of signal amplification. (A) Schematic representation of experimental groups. (B, C, D) Comparison of signal intensity of FAM-labeled DM1 in three labeling groups. SNR: signal-to-noise ratio. (E, F, G) Comparison of signal intensity of PE-Cy5.5 in three labeling groups. Streptavidin-PE-Cy5.5 bound biotin-DM2 conjugates or biotin-DM2-alginate conjugates. (B, C) Flow cytometry analysis of FAM signal. (E, F) Flow cytometry analysis of PE-Cy5.5 signal. (D, G) Fluorescence live cell imaging and corresponding line profiles. No significance (ns): p>0.05; *: p<0.05; **: p<0.01.

FIG. 2 demonstrates signal reversibility in the presence of triggering cDNA. (A) Schematic illustration of signal reversal. (B) Line profiles of fluorescent live cell images that are the inset images of panel E and panel H. (C, D) Flow cytometry analysis of FAM signal and (F, G) PE-Cy5.5 signal. (E) Fluorescence live cell imaging FAM signal and (H) PE-Cy5.5 signal. T_{DI}: triggering cDNA of DI; T_{DM1}: triggering cDNA of DM1; T_{DI/DM1}: T_{D1}+T_{DM1}. No significance (ns): p>0.05; *: p<0.05; **: p<0.01; ***: p<0.001.

FIG. 3 demonstrates signal reversibility in the presence of alginate lyase and DNAse. (A, B) Flow cytometry analyses of FAM signal. (C) Fluorescence live cell imaging for examination of FAM signal. (D, E) Flow cytometry analyses of PE-Cy5.5 signal. (F) Fluorescence live cell imaging for examination of PE-Cy5.5 signal. T_{DNA}: triggering cDNA of DI and DM1. T_{DNA}+(i.e., T_{DNA}+alginase): triggering cDNA of DI and DM1 coupled with alginate lyase. No significance (ns): p>0.05; *: p<0.05; **: p<0.01; ***: p<0.001.

FIG. 4 shows an evaluation of bidirectional cell imaging of the biomarker VCAM-1 using an FITC-labeled anti-VCAM-1 antibody (“FITC-Antibody”) or DI-conjugated anti-VCAM-1 antibodies in combination with DM1 and DM2-Alginate conjugates (“HCR”). The nanostructures were then dissociated using a combination of T_{DI}, T_{DM1}, and alginate lyase (“Triggered”). (a, b, c) Bright field (BF) and fluorescence microscopy images of cells labeled with the different methods. (a) Direct antibody labeling; (b) signal amplification; and (c) bidirectional signal amplification (i.e., signal amplification followed by treatment with the depolymerization agents). Scale bar: 50 μm.

FIG. 5 demonstrates that an initiator DNA (DI) is required to form DNA polymers comprising DM₁ and DM₂. (a) Schematic illustration of how DI, DM₁, and DM₂ interact to form a DNA polymer. The letters indicate regions that undergo hybridization (e.g., i-i* and j-j*). (b) A gel demonstrating that DNA polymers comprising DM₁ and DM₂ are formed in the presence of DI.

### DETAILED DESCRIPTION

The present invention is based on the inventors' development of improved, reversible methods of signal amplification. In these methods, nucleic acid-alginate conjugates are used amplify signals much more effectively than conventional methods. The methods and compositions of this disclosure are particularly advantageous because (i) detectable labels can be removed from a sample without the need for damaging proteases, and (ii) the methods can be adapted for use with either living or fixed cells and tissues. Furthermore, polymerase chain reaction is not involved.

The present invention provides methods for reversibly detecting a target analyte in a sample. The methods comprise: (a) contacting the sample with a probe that comprises an initiator single-stranded DNA molecule (ssDNA) conjugated to a targeting agent that binds to the target analyte; (b) washing the sample to remove unbound probe; (c) contacting the probe-target analyte complex with: (i) a first DNA hairpin comprising (1) a first portion that is complementary to both a part of the initiator ssDNA and a part of a second DNA hairpin, and (2) a second portion that is complementary to a part of a first portion of the second DNA hairpin; and (ii) the second DNA hairpin comprising a first portion that is complementary to a part of the second portion of the first DNA hairpin and a second portion that is complementary to a part of the first portion of the first DNA hairpin; wherein either the first hairpin or the second hairpin is linked to an alginate; and wherein the initiator ssDNA, the first DNA hairpin, and the second DNA hairpin undergo hybridization chain reaction (HCR) when in contact, thereby forming a nanoscaffold attached to the targeting agent; and (d) contacting the nanoscaffold of step (c) with a detectable label that binds to the alginate; and (e) detecting the detectable label.

As used herein, the term “target analyte” refers to the molecule of interest to be detected in the sample. The target analyte can be any molecule for which there exists a naturally or artificially prepared specific binding member (i.e., targeting agent). Suitable target analytes include, for example, a DNA, RNA, protein, peptide, amino acid, antibody, carbohydrate, lipid, hormone, steroid, toxin, vitamin, drug, bacterium, virus, or cell.

As used herein, the term “contacting” refers to a process in which two or more molecules or two or more components of the same molecule or different molecules are brought into physical proximity such that they are able undergo an interaction. Molecules or components thereof may be brought into contact by combining two or more different components containing molecules, for example by mixing two or more solution components, preparing a solution comprising two or more molecules such as target, candidate or competitive binding reference molecules, and/or combining two or more flowing components.

Alternatively, molecules or components thereof may be contacted combining a fluid component with molecules immobilized on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of media, including liquids, solutions, colloids, suspensions, emulsions, gels, solids, membrane surfaces, glass surfaces, polymer surfaces, vesicle samples, bilayer samples, micelle samples and other types of cellular models or any combination of these.

In step (a), the target analyte is bound by a probe that comprises an initiator single-stranded DNA molecule (ssDNA) conjugated to a targeting agent that binds to the target analyte.

As used herein, the term “initiator single-stranded DNA molecule (ssDNA)” refers to a single-stranded DNA molecule that is complementary to a first portion of a DNA hairpin (i.e., the first DNA hairpin), such that binding of the initiator ssDNA to the DNA hairpin causes the DNA hairpin to unfold into a linearized structure. This binding interaction between the initiator ssDNA and the first DNA hairpin is used to initiate hybridization chain reaction (HCR) in the present methods. Preferably, the initiator ssDNA has a linear structure with one functional domain.

In the methods of the present invention, the initiator ssDNA is conjugated to a targeting agent. As used herein, a “targeting agent” is an agent that specifically binds to the target analyte. Suitable targeting agents include, for example, proteins (e.g., antibodies), nucleic acids (e.g., aptamers and complementary sequences), and small molecules (e.g., ligands). In some embodiments, the targeting agent is an antibody or a nucleic acid aptamer that specifically binds to the target analyte. In a preferred embodiment, the targeting agent is an antibody or binding fragment thereof.

As used herein, the terms “conjugated” and “linked” are used interchangeably to refer to a strong attachment of a first molecule to a second molecule. Conjugated molecules may be attached via covalent or high strength non-covalent (e.g., biotin-streptavidin) interactions.

In step (b), the sample is washed to remove unbound probe. Suitable wash reagents include, without limitation, physiological buffers, phosphate buffered saline, or other solutions that do not damage cells. Suitable wash solutions are known and understood by one skilled in the art and contemplated herein depending on the method of detection and the sample (e.g., cells, tissues, etc.).

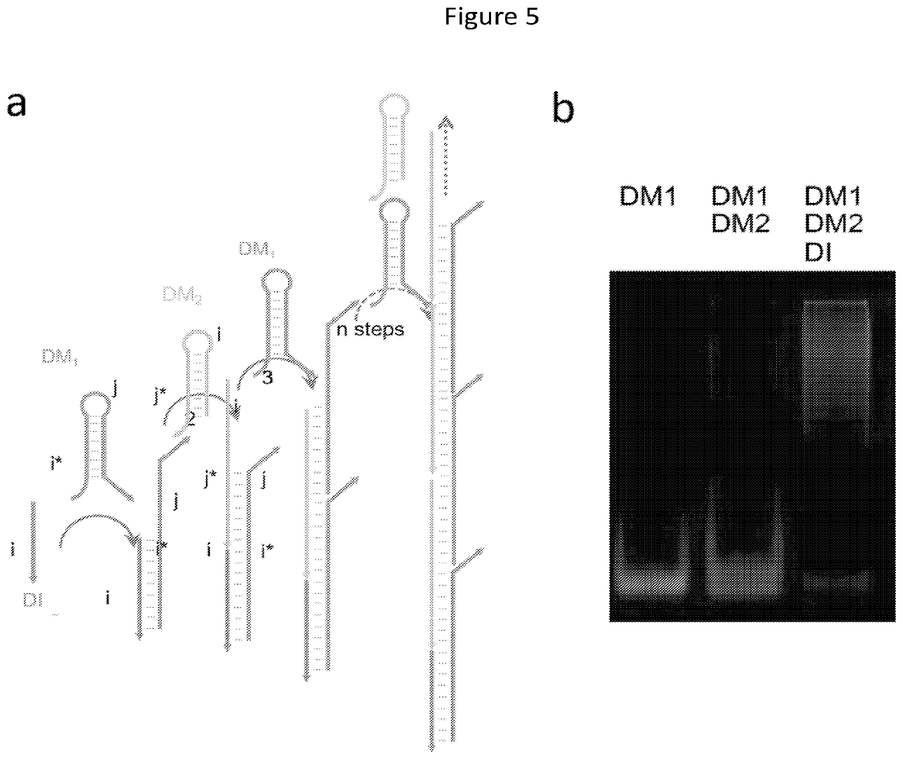

In step (c), the probe-target analyte complex is contacted with two DNA hairpins that are capable of undergoing hybridization chain reaction (HCR) in the presence of the initiator ssDNA. During HCR, hybridization with the initiator ssDNA (DI) opens the hairpin structure of the first hairpin DNA (DM1), thereby linearizing a segment that is complementary to a first portion of the second hairpin DNA (DM2). Hybridization of DM2 to this exposed portion of the DM1 linearizes DM2, exposing a segment of DM2 that is complementary to the first portion of the DM1 (and identical to the intiator sequence). This segment serves as a new DNA initiator, hybridizing with DM1 to initiate subsequent cycles of polymerization. See FIG. 5A for a schematic depiction of this process. Thus, the first DNA hairpin used with the present invention must comprise (1) a first portion that is complementary to both a part of the initiator ssDNA and a part of the second DNA hairpin, and (2) a second portion that is complementary to a part of a first portion of the second DNA hairpin; and, and the second DNA hairpin must comprise a first portion that is complementary to a part of the second portion of the first DNA hairpin and a second portion that is complementary to a part of the first portion of the first DNA hairpin (and is identical to the intiator sequence of the intiator ssDNA). The binding of the initiator DI to the first DM1 results in an overhang in which DM2 may hybridize to this overhang. Once DM1 and DM2 hybridize, there is left an overhang on the DM2 strand in which another DM1 may hybridize and so on to polymerize into a double stranded DNA molecule making up the nanoscaffold.

The term “overhang,” as used herein, refers to terminal non-base pairing nucleotide(s) resulting from one strand or region extending beyond the terminus of the complementary strand to which the first strand or region forms a duplex. One or more polynucleotides that are capable of forming a duplex through hydrogen bonding can have overhangs. The single-stranded region extending beyond the 3′ end of the duplex is referred to as an overhang.

The terms “hybridize” and “hybridization” as used herein refer to the association of two single-stranded nucleic acids biding non-covalently to form a double-stranded nucleic acid (stable duplex). Nucleic acids hybridize due to a variety of well-characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.). Complementary sequences in the nucleic acids pair with each other to form a double helix, this resulting double-stranded nucleic acid is sometimes referred to as a “hybrid”. One of skill in the art will understand that “hybridization” as used herein does not require a precise base-for-base complementarity. That is, a duplex can form, between two nucleic acids that contained mismatched base pairs. As used herein, the term “complementary” refers to a nucleic acid that forms a stable duplex with its “complement”. For example, nucleotide sequences that are complementary to each other have mismatches at less than 20% of the bases, at less than about 10% of the bases, preferably at less than about 5% of the bases, and more preferably have no mismatches.

A first oligonucleotide anneals with a second oligonucleotide with “high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known.

An exemplary set DNA sequences that can be used as a ssDNA (DI), first DNA hairpin (DM1), and second DNA hairpin (DM1) are provided in Table 1. Thus, in some embodiments, the single-stranded DNA molecule comprises SEQ ID NO:1 or SEQ ID NO:2, the first DNA hairpin comprises SEQ ID NO:3, and the second DNA hairpin comprises SEQ ID NO:4.

This HCR reaction forms a nanoscaffold attached to the targeting agent. As used herein, the term “nanoscaffold” is used to refer to a product comprising polymerized nucleic acid-alginate conjugates that is formed by hybridization chain reaction. Preferably, the nanoscaffold comprises a plurality of repeating units, each of which comprises an alginate molecule that is linked to a plurality of detectable labels. The nanoscaffolds of the present invention comprise multiple alginate molecules (N). Each alginate can be conjugated to multiple detectable labels or to biotin (M). When biotin is used, each alginate can bind to multiple detectable label-streptavidin conjugates (O). In such cases, each target analyte will be linked to N*M or N*M*O detectable labels.

In the present methods, either the first hairpin or the second hairpin can be linked to an alginate. However, it is not preferably that the alginate be linked to both the first hairpin and the second hairpin as this may cause excessive steric hindrance. In the Examples, the inventors conjugated the alginate to the second DNA hairpin (i.e., DM2Thus, in some embodiments, the second DNA hairpin is linked to the alginate conjugated to a binding agent.). In another embodiment, the alginate may be conjugated to the first DNA hairpin (i.e., DM1).

In step (d), the nanoscaffold of step (c) is contacted with a detectable label that binds to the alginate. As used herein, the term “detectable label” is used to refer to any a molecule or particle that can be detected. Suitable detection labels include, without limitation, epitope tags, detectable markers, radioactive markers, and nanoparticles. Suitable epitope tags are known in the art and include, but are not limited to, 6-Histidine (His), hemagglutinin (HA), cMyc, GST, Flag tag, V5 tag, and NE-tag, among others. Suitable detectable markers include luminescent markers, fluorescent markers or fluorophores (e.g., fluorescein, fluorescein isothiocyanate, rhodamine, dichlorot[pi]azinylamine fluorescein, green fluorescent protein (GFP), red fluorescent protein (RFP), blue fluorescent dyes excited at wavelengths in the ultraviolet (UV) part of the spectrum (e.g., AMCA (7-amino-4-methylcoumarin-3-acetic acid); Alexa Fluor 350), green fluorescent dyes excited by blue light (e.g., FITC, Cy2, Alexa Fluor 488), red fluorescent dyes excited by green light (e.g., rhodamines, Texas Red, Cy3, Alexa Fluor dyes 546, 564 and 594), or dyes excited with infrared light (e.g., Cy5), dansyl chloride, and phycoerythrin), or enzymatic markers (e.g., horseradish peroxidase, alkaline phosphatase, beta-galactosidase, glucose-6-phosphatase, and acetylcholinesterase). Suitable radioactive markers include, but are not limited to, 1251, 1311, 35S or 3H. Suitable nanoparticles, including metal nanoparticles and other metal chelates, are known in the art and include, but are not limited to, gold nanoparticles (ACSNano, Vol. 5, No. 6, 4319-4328, 2011), quantum dots (Nanomedicine, 8 (2012) 516-525), magnetic nanoparticles (Fe3O4), silver nanoparticles, nanoshells, and nanocages.

In some embodiments, the dateable label is a fluorophore. As used herein, the term “fluorophore” includes molecules that absorb a photon of a wavelength and emit a photon of another wavelength. This term also includes molecules that are inherently fluorescent or demonstrate a change in fluorescence upon binding to a biological compound or metal ion, or upon metabolism by an enzyme (i.e., fluorogenic). Numerous fluorophores are known to those skilled in the art and include, without limitation, coumarins, acridines, furans, dansyls, cyanines, pyrenes, naphthalenes, benzofurans, quinolines, quinazolinones, indoles, benzazoles, borapolyazaindacene, oxazines and xanthenes, with the latter including fluoresceins, rhodamines, rosamines and rhodols. Exemplary fluorophores for compositions of this disclosure include, without limitation, fluorescein, FAM (6-fluorescein amidite), PE-Cy5.5, sulforhodamine 101, pyrenebutanoate, acridine, ethenoadenosine, eosin, rhodamine, 5-(2′-aminoethyl)aminonaphthalene (EDANS), fluorescein isothiocyanate (FITC), N-hydroxysuccinimidyl-1-pyrenesulfonate (PYS), tetramethylrhodamine (TAMRA), Rhodamine X, Cy5, and erythrosine. In some embodiments, the fluorophore is selected from fluorescein, FAM (6-fluorescein amidite), sulforhodamine 101, pyrenebutanoate, acridine, ethenoadenosine, eosin, rhodamine, 5-(2′-aminoethyl)aminonaphthalene (EDANS), fluorescein isothiocyanate (FITC), N-hydroxysuccinimidyl-1-pyrenesulfonate (PYS), tetramethylrhodamine (TAMRA), Rhodamine X, Cy5, and erythrosine.

In step (e), the detectable label is detected. Appropriate methods of detection will be dictated by the detectable label that is employed. For example, a fluorescent label may be visualized using fluorescent microscopy. Alternatively, the detectable label may be detected by flow cytometry or used in flow cytometric cell sorting techniques, which are well understood by one skilled in the art.

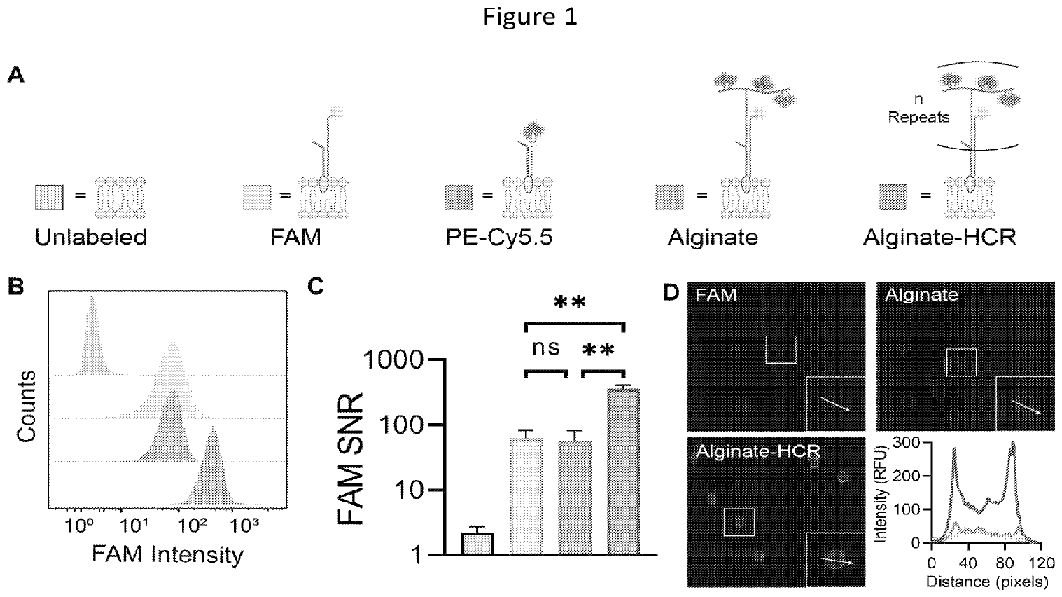

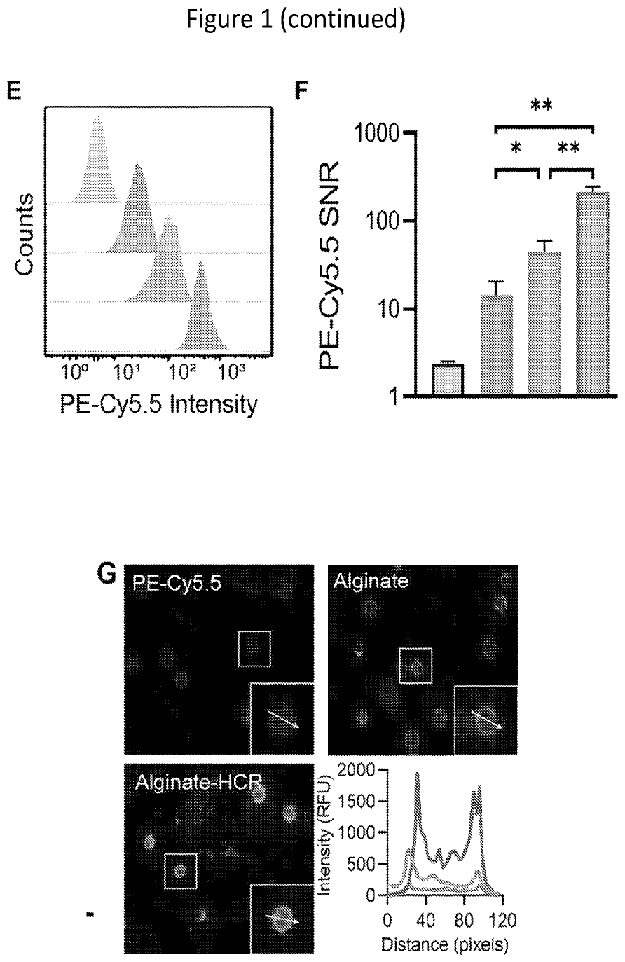

The methods of the present invention are designed to increase the signal produced by the detectable label (e.g., amplify the signal) as compared to the signal produced in the absence of HCR. In the Examples, the inventors demonstrate that their method increases the signal intensity by 5-fold (for FAM; see FIG. 1) and by 14-fold (for PE-Cy5.5; see FIG. 1). Thus, in some embodiments, the methods increase the signal by at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold as compared to the signal produced in the absence of HCR. This ability to amplify a signal is important especially in cases in which there is limited samples. For examples, when measuring a biomolecule on a cell, if there are very few cells within the sample, the present methods allow for signal amplification that would allow for the detection of a signal even if there are very few biomolecules and/or very few cells within the sample.

The methods of the present invention are also designed to be reversible. As used herein, the terms “reversible” and “bidirectional” are used interchangeably to refer to the ability to remove most or substantially all of the detectable label from the sample. In some embodiments, the removal of the detection signal results in less than 25% of the original detection signal for the sample, alternatively less than 15%, alternatively less than 10%, alternatively less than 5%, alternatively less than 2% of the original detection signal is measured in the sample after the removal of the detectable signal. Preferably, methods will reduce the signal by at least an order of magnitude (i.e., to less than 10% of the original detection signal). Thus, in some embodiments, the methods further comprise (f) removing the detectable label from the sample by contacting the sample with a depolymerization agent. In some embodiments, removing the detectable label involves removing most or substantially all of the DNA nanoscaffold.

As used herein, the terms “depolymerizing agent,” “depolymerization agent,” “trigger molecule,” and “trigger sequence” all refer to an agent that can depolymerize the hybridized DNA sequences, or in other words, be used to dissociate components of the nanoscaffold, thereby degrading the nanoscaffold. The term “depolymerize” or “depolymerization” as used herein includes the process of two DNA sequences attaching together through hybridization such that one of the DNA sequences which was previously hybridized in a polymerization of DNA oligonucleotides in the nanoscaffold is now hybridized to a single oligonucleotide and no longer is a participant in the dsDNA polymerization structure within the nanoscaffold. In other words, the depolymerizing agent is an agent that unhybridizes the double stranded DNA formed from the DM1 and DM2 molecules.

In some embodiments, the depolymerization agent is a complementary DNA (cDNA), an alginate lysase, a DNase or combinations thereof. In some embodiments, a combination of two or more depolymerizing agents is used, e.g., two cDNA, a cDNA and an alginate lyase, a cDNA and a DNase, an alginate lysase and a DNase, or other suitable combinations thereof.

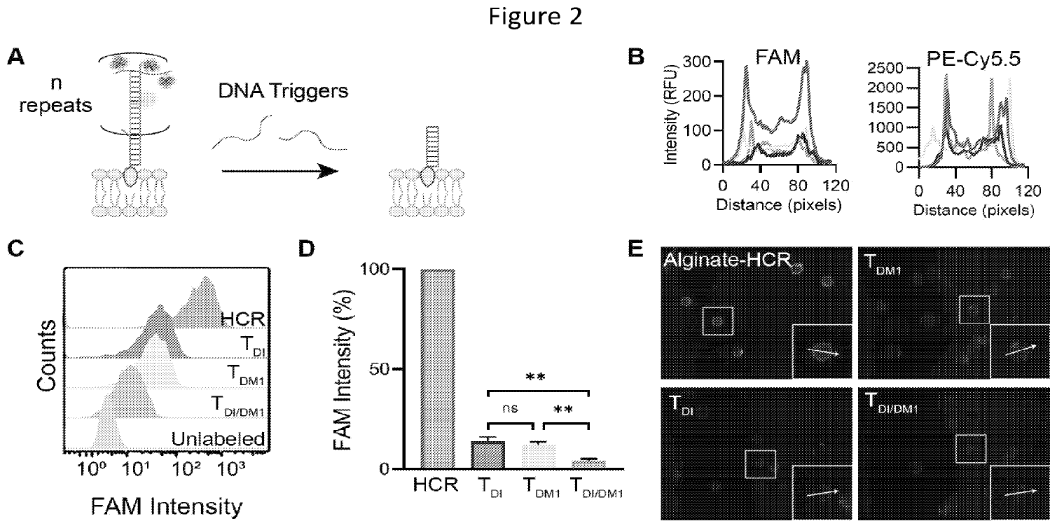

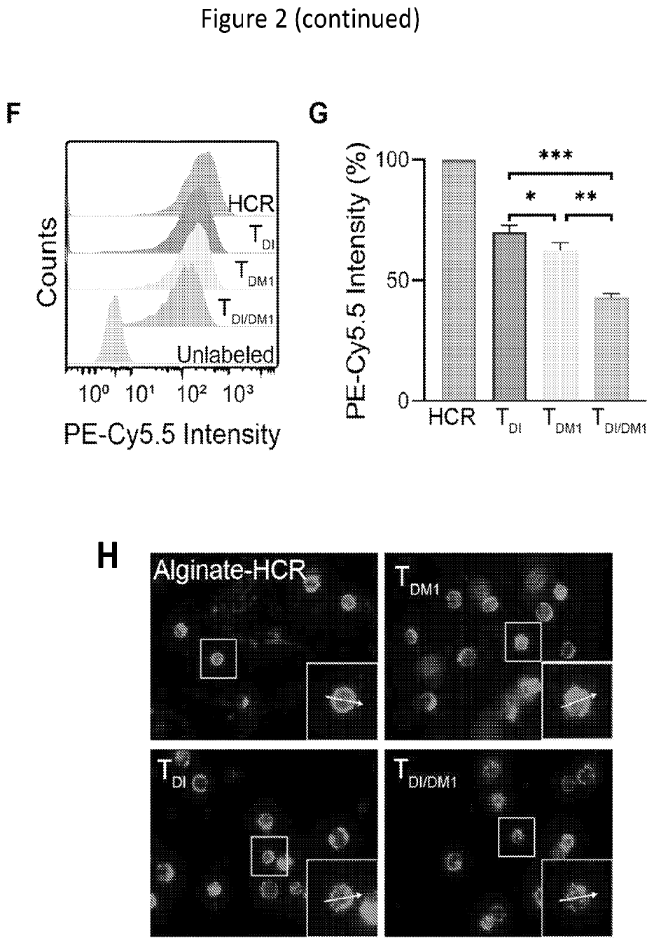

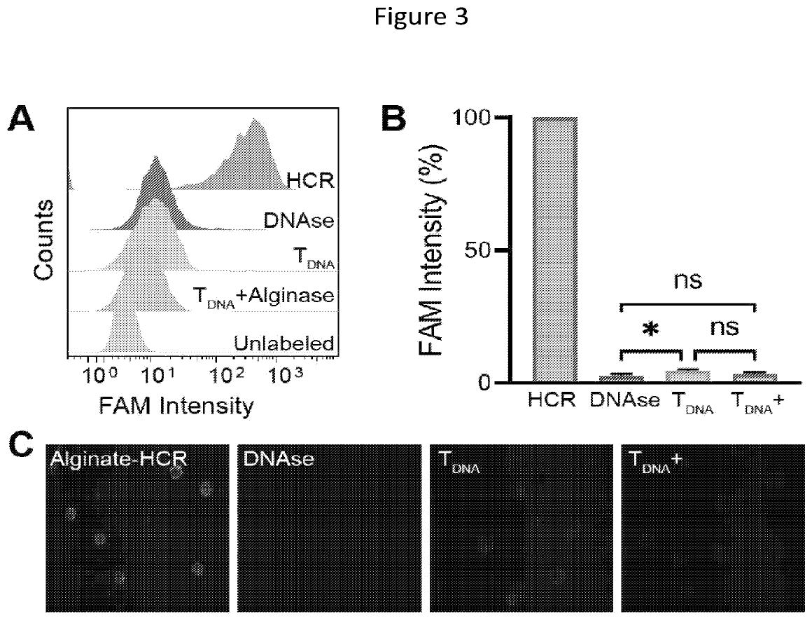

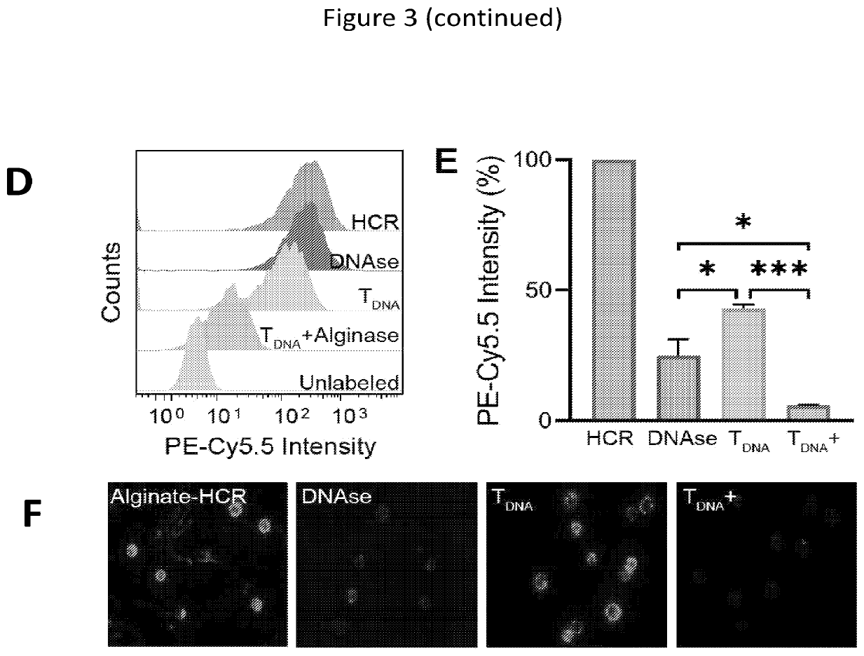

In the Examples, the inventors demonstrate that their HCR labeling can be reversed using a complementary DNA (cDNA) that is complementary to one of the nanoscaffold components (i.e., the initiator ssDNA or a hairpin DNA), an alginate lyase, or a DNase (see FIGS. 2 and 3). Thus, any of these reagents may be used as a depolymerizing agent in the methods of the present invention.

The depolymerization agent may be a cDNA that is complementary to a portion of any of the HCR components (i.e., the initiator ssDNA or a hairpin DNA), and is designed to bind to a single-stranded overhang region (i.e., a toehold) within the original strand (e.g. the initiator-ssDNA or a hairpin DNA). In some embodiments, the depolymerization agent comprises a cDNA that is complementary to at least a portion of the initiator ssDNA, a cDNA that is complementary to at least a portion of the first DNA hairpin, a cDNA that is complementary to at least a portion of the second DNA hairpin, or any combination thereof. For example, in some embodiments, the cDNA comprises SEQ ID NO:6 (TDI), SEQ ID NO:7 (TDMI), or both SEQ ID NO:6 and SEQ ID NO:7 (TD1/DMI). Binding of the cDNA causes toehold-switch mediated displacement of its cognate sequence.

The inventors found that using a combination of both cDNAs and an alginate lyase was the effective for removing the detectable label from the sample. Thus, in some embodiments, the depolymerization agent comprises a cDNA and an alginate lyase. An alginate lyase (also referred to as an “alginate”) is an enzyme that degrades alginate. Any alginate lyase can be used with the present invention including, for example, those that have been isolated from algae, marine mollusks, marine and terrestrial bacteria, viruses, and fungi (Bioengineered. 6(3): 125-131, 2015, incorporated by reference in its entirety regarding alginate lysase). Notably, alginate lyase is not a naturally occurring material in mammalian cell systems or tissues and, thus, can be used without causing damage to mammalian cells or tissues.

In some embodiments, the depolymerizing agent comprises DNA analogues (e.g., nucleopeptides and interlocked DNA). DNA analogues can interact with the DNA nanoscaffolds in a similar fashion cDNAs, but may have a higher binding affinity for DNA initiator sequences or DNA monomers. Moreover, DNA analogues are usually stable against DNase. Accordingly, it is possible to use DNA analogues together with the combination of DNAse and alginate lyase described herein.

The reversibility of the present methods is advantageous for several downstream applications. For example, this method can be used to label and sort live functional cells such as stem cells or CAR-T cells. Once the cells have been isolated based on cell surface expression of certain markers, the label can be removed, which ensures that the label will not interfere with therapeutic applications. Thus in some embodiments, the sample are engineered human cells, such as CAR-T cells, or progenitor cells, for example, induced pluripotent stem cells or other derivatives of stem cells that may be used therapeutically. The methods described herein can be used to sort live functional cells and then remove the label from the cells before administration to a subject, thus lowering any adverse effects that may accompany additional detection agents used on the cells for sorting.

Additionally, the reversibility of these methods allows them to be used to label multiple target analytes sequentially within a sample. This ability is particularly useful when the sample is limited in supply. Thus, in some embodiments, the methods further comprise: (g) repeating steps (a)-(e) using a different targeting agent to detect a different target analyte. In some embodiments, the sample is a cell sample. In some embodiments, the cell sample is a biopsy, for example a cancer biopsy.

In some embodiments, the sample is taken from a subject. In some embodiments, the sample is a biopsy. In other embodiments, the sample is a tissue or organ sample. In some embodiments, the sample may be a sample taken during surgery of tissue or organ. Other suitable samples also include body fluids, for example, blood, plasma, urine, sputum, saliva, mucus, etc. taken from a subject.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human.

In the methods of the present invention, alginate is conjugated to one of the DNA hairpins. Alginate (alginic acid) is a linear unbranched polysaccharide composed of (1-4)-linked β-D-mannuronate (M) and α-L-guluronate (G) monomers that is found in the cell walls of brown algae. Along its polymeric chain, the monomers are organized in blocks of M, G, and M-G/G-M sequences. Alginate is known for biocompatibility with cells and tissues. In some embodiments, the alginate used with the present invention is a branched alginate (i.e., branched alginic acid (bAlg)), which can be synthesized using, for example, an amine-terminated branched polyethylene glycol such as amine-terminated 4-arm branched polyethylene glycol (4-arm PEG, 20,000 Da).

Although alginate is exemplified herein, those of skill in the art will appreciate that other polymers can be used in place of alginate. For instance, any azide-functionalized polymer capable of rapid hydrolysis in the presence of an enzyme can be directly substituted for alginate when click chemistry is used for the conjugation reaction. Alternative conjugation reactions can be used, which allows for the use of additional polymers in place of alginate. In embodiments that utilize an alternative polymer, any enzyme that degrades that polymer may be used as a depolymerization agent.

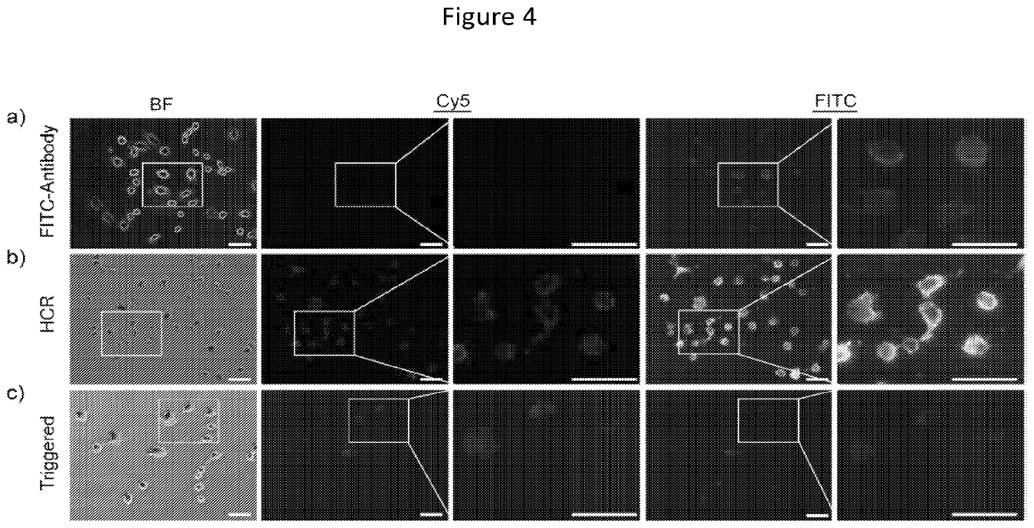

In the present methods, alginate is used as a platform onto which multiple detectable labels can be conjugated. In the Examples, the inventors used alginate that was directly conjugated to a detectable label (see FIG. 4) and alginate that was linked to a detectable label via a streptavidin-biotin interaction (see FIGS. 1-3). Thus, in some embodiments, the detectable labels are directly linked to the alginate. In other embodiments, the alginate is conjugated to a binding agent and the detectable label is conjugated to a binding partner, such that the detectable label binds to the alginate via the interaction of the binding agent and the binding partner. In certain embodiments, the binding agent is biotin and the binding partner is streptavidin. However, any suitable binding agent-binding partner pair may be utilized to link alginate to a detectable label. Suitable binding agent-binding partner pairs include, for example, avidin, streptavidin, or NeutrAvidin™ paired with biotin or desthiobiotin; cucurbit[7]uril (CB [7]) or β-cyclodextrin and ferrocene or its derivatives; and the like. Further, because alginate is negatively charged, it can form a complex with positively charged polymers such as cationic polymers carrying fluorophores for detection.

DNA hairpin-alginate conjugates can be prepared according to any appropriate method. As described in the examples, the method can comprise modifying alginate to incorporate reactive azide groups and then conjugating the nucleic acid to the azide-modified alginate via a click chemistry reaction. In some cases, the nucleic acid is conjugated to the azide-modified alginate and the alginate is subsequently biotinylated via two or more click chemistry reactions. Preferably, the click chemistry reaction is copper-free click chemistry to avoid the potential toxicity of copper catalysts. In some cases, copper-free-click chemistry is used to conjugate DBCO-modified DNA/biotin/fluorophore with azide-modified (N3) alginate chains. Azide-modification is a requirement for any polymer to be substituted directly for alginate in the copper-free-click chemistry reaction scheme.

The methods of the present invention may be applied to any sample that comprises a target analyte of interest. Suitable samples include, without limitation, patient samples, environmental samples, cell culture samples, and animal or plant tissue. In some cases, samples are obtained by swabbing, washing, or otherwise collecting biological material from a non-biological object such as a medical device, medical instrument, handrail, doorknob, etc.

In the Examples, the inventors demonstrated that the methods can be used to detect a biomolecule (i.e., VCAM-1) on the surface of a cell. Thus, in some embodiments, the sample comprises one or more cells. In some embodiments, the sample comprising live cells, either individually or within a tissue. In other embodiments, the sample comprises fixed cells.

A sample can be an unprocessed or a processed sample; processing can involve steps that increase the purity, concentration, or accessibility of components of the sample to facilitate the analysis of the sample. As nonlimiting examples, processing can include steps that reduce the volume of a sample, remove or separate components of a sample, solubilize a sample or one or more sample components, or disrupt, modify, expose, release, or isolate components of a sample. Nonlimiting examples of such procedures are centrifugation, precipitation, filtration, homogenization, cell lysis, binding of antibodies, cell separation, etc. For example, in some embodiments of the present invention, the sample is a blood sample that is at least partially processed, for example, by the removal of red blood cells, by concentration, or by selection of one or more cell (for example, white blood cells or pathogenic cells), etc. In one embodiment, the method is useful for detecting biomolecules in cells that are immobilized on a hydrogel.

Exemplary samples include a solution of at least partially purified nucleic acid molecules. The nucleic acid molecules can be from a single source or multiple sources, and can comprise DNA, RNA, or both. For example, a solution of nucleic acid molecules can be a sample that was subjected to any of the steps of cell lysis, concentration, extraction, precipitation, nucleic acid selection (such as, for example, poly A RNA selection or selection of DNA sequences comprising Alu elements), or treatment with one or more enzymes. The sample can also be a solution that comprises synthetic nucleic acid molecules.

In certain embodiments, the target analyte is a cell surface biomolecule. In other embodiments, the target analyte is an intracellular biomolecule. In embodiments in which the target analyte is intracellular, the methods further comprise, prior to step (a), fixing and permeabilizing the cells in the sample to allow the HCR reagents to access the target analyte. Methods of fixing and permeabilizing cells are well known in the art. For example, cells can be fixed using formalin, formaldehyde, or paraformaldehyde fixation techniques. In some cases, the tissue is formalin-fixed and paraffin-embedded (FFPE). Any fixative that does not affect antibody binding or nucleic acid hybridization can be utilized in the methods provided herein.

Samples may need to be modified in order to render the biomarker antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the biomarker more accessible for target binder. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Methods for detecting fluorescent molecules in a cell preparation are well known in the art. Such methods include but are not limited to detection using flow cytometry with or without flow associated cell sorting (FACS) and analysis, or fluorescent microscopy imaging. Additionally, in addition to detecting the signal, the methods described herein my measure a level of one or more target anaytes.

In cases in which the target analyte is a protein, methods of measuring levels of one or more proteins of interest in a biological sample include, but are not limited to, an immuneochromatography assay, an immunodot assay, a Luminex assay, an ELISA assay, an ELISPOT assay, a protein microarray assay, a Western blot assay, a mass spectrophotometry assay, a radioimmunoassay (MA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

As used herein, the term “biomolecule” or “biomarker” refers to any molecule that is of biological origin. This term encompasses deoxyribonucleic acid (DNA), ribonucleic acid (RNA), nucleotides, oligonucleotides, nucleosides, polynucleotides, proteins, peptides, polypeptides, antibodies, antigens, protein complexes, aptamers, haptens, combinations thereof, and the like.

In various embodiments, the protein form of the biomarkers is measured. In various embodiments, the nucleic acid form of the biomarkers is measured. In exemplary embodiments, the protein form is detected using an antibody.

When the antibody used in the methods of the invention is a polyclonal antibody (IgG), the antibody is generated by inoculating a suitable animal with a biomarker protein, peptide or a fragment thereof. Antibodies produced in the inoculated animal which specifically bind the biomarker protein are then isolated from fluid obtained from the animal. Biomarker antibodies may be generated in this manner in several non-human mammals such as, but not limited to goat, sheep, horse, rabbit, and donkey. Methods for generating polyclonal antibodies are well known in the art and are described, for example in Harlow, et al. (1998, In: Antibodies, A Laboratory Manual, Cold Spring Harbor, N.Y.).

When the antibody used in the methods of the invention is a monoclonal antibody, the antibody is generated using any well-known monoclonal antibody preparation procedures such as those described, for example, in Harlow et al. Given that these methods are well known in the art, they are not replicated herein. Generally, monoclonal antibodies directed against a desired antigen are generated from mice immunized with the antigen using standard procedures. Monoclonal antibodies directed against full length or peptide fragments of biomarker may be prepared using the techniques described in Harlow, et al. (1998, In: Antibodies, A Laboratory Manual, Cold Spring Harbor, N.Y.).

In some embodiments, the sample is washed to remove excess first DNA hairpin and second DNA hairpin before step (d). Any suitable wash solution known in the art can be used.

Kits

In another embodiment, the present invention provides kits for detecting a target analyte in a sample. The kits comprise: (a) a probe that comprises an initiator single-stranded DNA molecule (ssDNA) conjugated to a targeting agent that binds to the target analyte; (b) a first DNA hairpin comprising (1) a first portion that is complementary to both a part of the initiator ssDNA and a part of a second DNA hairpin, and (2) a second portion that is complementary to a part of a first portion of the second DNA hairpin; (c) a second DNA hairpin comprising a first portion that is complementary to a part of the second portion of the first DNA hairpin and a second portion that is complementary to a part of the first portion of the first DNA hairpin; wherein the first DNA hairpin or second DNA hairpin is linked to alginate; and (d) a detectable label that binds to the alginate or is conjugated to the alginate.

In some embodiments, the alginate provided with the kit is conjugated to a binding agent and the detectable label provided with the kit conjugated to a binding partner such that the detectable label binds to the alginate via the interaction of the binding agent and the binding partner. In some embodiments, the binding agent is biotin and the binding partner is streptavidin.

In some embodiments, the kits further comprise a depolymerization agent. In some embodiments, the depolymerization agent is selected from a complementary DNA (cDNA) molecule, alginate lyase, DNase, and any combination thereof. In certain embodiments, the depolymerization agent comprises a cDNA that is complementary to at least a portion of the initiator ssDNA, a cDNA that is complementary to at least a portion of the first DNA hairpin, a cDNA that is complementary to at least a portion of the second DNA hairpin, or any combination thereof. In some embodiments, two or more depolymerization agents are provided.

In some cases, the kits may also contain one of more of the following: a biological sample preservative or additive, such as an agent that prevents degradation of nucleic acid (e.g., formaldehyde), a reaction buffer in which the HCR components and the biological sample are mixed, one or more reagents for detecting a colorimetric signal, a negative control sample, a positive control sample, one or more reaction containers, such as tubes or wells, and an instruction manual.

“Percentage of sequence similarity” or “percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user. The term “substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially” of and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The invention will be more fully understood upon consideration of the following non-limiting Examples. The examples herein use several specific sequences, but it will be appreciated by one of ordinary skill in the art that other sequences are readily amenable for use in the disclosed methods.

### Examples

### Example 1—Bidirectional Signal Amplification for Cell Labeling and Imaging Via Reversible Hybridization Chain Reaction

This Example describes the development of a biomolecular system for bidirectional signal amplification using hybrid DNA-polymer conjugates, triggering complementary DNA (cDNA) and an alginate lyase that is neither a protease nor a DNAse. As described herein, a supramolecular DNA-alginate nanoscaffold was synthesized in situ on the surface of a target cell. This nanoscaffold has multiple repeating units, each of which has an alginate molecule that carries numerous biotin molecules as binding sites for binding to fluorophore-conjugated streptavidin. As the nanoscaffold is made of DNA, it can be depolymerized using cDNA. Moreover, as the polymer is alginate, the branches of the nanoscaffold can be degraded using alginate lyase that does not hydrolyze either proteins or nucleic acids. Thus, this molecular system enables bidirectional signal amplification.

Methods

Hybridization Chain Reaction

A schematic showing how hybridization chain reaction (HCR) can be used to form branched DNA polymers is shown in FIG. 5A. This process involves three molecules: a DNA initiator (DI) and two DNA monomers (DM₁ and DM₂). DI has a linear structure and one functional domain, which is labeled with i. The DNA monomers form hairpin stem-loops. DM₁ has two domains: i* and j; and DM₂ has two domains: i and j*. During polymerization, hybridization with DI opens the hairpin nanostructure of DM₁ to form an i-i* double helix with j left as a linear segment. The linear j domain further reacts with the j* domain of DM₂ to form a j-j* double helix, linearizing DM₂ and exposing linear segment i. The linear segment i functions as a new DNA initiator, hybridizing with DM₁ to initiate subsequent cycles of polymerization, thereby forming a supramolecular DNA nanomaterial.

A gel electrophoresis experiment was performed to test whether a mixture of only DM₁ and DM₂ could form a supramolecular DNA nanomaterial, or whether DI was required to initiate the reaction (FIG. 5B). DM₁ and DM₂ comprise complementary regions that are 18 base pairs in length. With this high level of intramolecular hybridization, we suspected that DM₁ and DM₂ may form stable, locked structures at 37° C. As expected, the results show that the mixture of DM₁ and DM₂ did not react in the absence of DI (FIG. 5B, Lane 2), but were able to form a supramolecular DNA nanomaterial in the presence of DI (FIG. 5B, Lane 3).

TABLE 1
DNA sequences

| DNA | | SEQ ID |
|---|---|---|
| Label | Sequence (5′→3′) | NO: |
| DI- | CCCTCACTCA CCTCATCCCACTCCTAC | 1 |
| Cholesterol | CTAAACC AAAAAAAAAA/3CholTEG/ | |
| DI-NH₂ | CCCTCACTCA CCTCATCCCACTCCTAC | 2 |
| | CTAAACC AAAAA/3AmMO/ | |
| DM1-FAM | GGTTTAGGTAGGAGTGGGATGAGG CCA | 3 |
| | AATCCTCATCCCACTCCTACCACTCACT | |
| | CCC/36-FAM/ | |
| DM1-Cy5 | GGTTTAGGTAGGAGTGGGATGAGG CCA | 3 |
| | AATCCTCATCCCACTCCTACCACTCACT | |
| | CCC/36-Cy5/ | |
| DM2-NH2 | /5AmMC6/AAAAA CCTCATCCCACTCC | 4 |
| | TACCTAAACC GGTAGGAGTGGGATGAG | |
| | GATTTGG | |
| CS_{DI}- | TTTTT GGTTTAGGTAGGAGTGGGATGA | 5 |
| Biotin | GG/3Bio/ | |
| T_{DI} | TTTTT GGTTTAGGTAGGAGTGGGATGA | 6 |
| | GG TGAGTGAGGG | |
| T_{DM1} | GGGAGTGAGT GGTAGGAGTGGGATGAG | 7 |
| | GATTTGG | |

Materials and Instrumentation

DNA sequences (Table 1) were purchased from Integrated DNA Technologies (Coralville, Iowa). Dibenzocyclooctyne (DBCO) reagents, including DBCO-PEG₄-NHS ester, DBCO-AlexaFluor488 and DBCO-PEG₄-Biotin were purchased from Click Chemistry Tools (Scottsdale, Ariz.). Sodium alginate, O-(2-Aminoethyl)-0′-(2-azidoethyl)pentaethylene glycol (NH₂-PEG₆-N₃), 2-(N-Morpholino)ethanesulfonic acid sodium salt (MES sodium salt), N-hydroxysuccinimide (NETS), 1-ethyl-3-(3-dimethylaminopropyl)carbodiimide hydrochloride (EDC), sodium hydroxide (NaOH), anhydrous dimethyl siloxane (DMSO) and Dulbecco's Modified Eagle's Medium (DMEM) were purchased from Sigma-Aldrich (St. Louis, Mo.). Acetone and sodium bicarbonate (NaHCO₃) were purchased from Fisher Scientific (Pittsburgh, Pa.). Dulbecco's phosphate buffered saline (DPBS), fetal bovine serum (FBS), and Roswell Park Memorial Institute (RPMI)-1640 medium were purchased from Gibco (Gaithersburg, Md.). Untagged and FITC-tagged VCAM-1 (CD106) monoclonal antibodies were purchased from Invitrogen (Carlsbad, Calif.). A protein-oligonucleotide conjugation kit containing succinimidyl 4-formylbenzoate (S-4FB), succinimidyl 6-hydrazinonicotinate acetone hydrazone (S-HyNic), and 2-Hydrazinopyridine (2-HP) was purchased form TriLink Biotechnologies (San Diego, Calif.).

Flow cytometry analyses were performed using a Guava easyCyteTM flow cytometer (Millipore). Brightfield and fluorescent cell images were captured using an Olympus IX73 inverted microscope system. UV-Vis spectrophotometry analyses were conducted using a Thermo Scientific NanoDrop 2000 spectrophotometer.

Cell Culture Conditions

Cell labeling experiments were performed using human acute lymphoblastic leukemia (CCRF-CEM) and mouse endothelial (C166) cell lines purchased from ATCC (Manassas, Va.). Human acute lymphoblastic leukemia cells (CCRF-CEM) were maintained in RPMI-1640 medium supplemented with 10% fetal bovine serum. Mouse endothelial cells (CCRF-CEM) were maintained in MEM medium supplemented with 10% fetal bovine serum. Cells were incubated at 37° C. with an atmosphere of 5% CO₂ and 95% relative humidity).

### Preparation of Azide-Modified Alginate (Alginate-N₃)

Azide-modified alginate (Alginate-N₃) was prepared through NHS/EDC coupling of carboxyl groups. 100 mg of sodium alginate was dissolved in 10 mL of MES buffer (50 nM, pH=5). 28 mg NHS, 232 mg EDC, and 56 μL NH₂-PEG₆-N₃ (mM) were added to the sodium alginate solution and stirred for 30 minutes. The pH was adjusted to 7.5 with 6M NaOH and the reaction proceeded overnight at room temperature. Algiante-N₃ was purified of unreacted reagents by dialysis (10 kDa MWCO) against ddH₂O, followed by precipitation in chilled acetone.

### Preparation of DBCO-Modified DM₂ (DM₂-DBCO)

DM₂-DBCO conjugates were formed through amine-reactive crosslinker chemistry. DM₂—NH₂ was dissolved in ddH₂O to 1 mM. A 30 mM solution of DBCO-PEG₄-NHS ester was prepared. 100 μL DM₂—NH₂ was mixed with 25 μL of DBCO-PEG₄-NHS ester in a NaHCO₃ buffer (50 nM) and allowed to react for 6 hours at room temperature. This reaction was repeated a total of 3 times. Excess DBCO-PEG₄-NHS ester linkers were removed by centrifugal filtration (3 kDa MWC). The concentration of purified DM₂-DBCO was determined by UV-Vis spectrophotometric analysis of the DBCO chromophore (λ=310 nm).

### Preparation of DM₂-Alginate Conjugates

Alginate-N₃ and DM₂-DBCO were covalently crosslinked via a copper-free click chemistry reaction. DM₂-DBCO was mixed with Alginate-N₃ (1% w/v) at a 3:1 molar ratio and reacted for 2 hours. DM₂-alginate was collected and purified of excess DM₂-DBCO by centrifugal filtration (100 kDa MWCO). DM₂-Alginate was further modified with either biotin or fluorescent molecules. Either DBCO-PEG₄-Biotin, DBCO-Cy5 or DBCO-AlexaFluor488 was mixed with DM₂-Alginate conjugates at a 3:1 ratio and reacted for 2 hours. Sample was collected and purified by centrifugal filtration (100 kDa MWCO).

### Preparation of DI-Antibody Conjugates

DI-Antibody conjugates were generated according to the protein-oligonucleotide conjugation kit protocol. Amine-modified DNA initiator (DI-NH₂) sequences were first modified with amino-reactive S-4FB. UV-Vis absorbance was used to determine the volume of 30 OD₂₆₀ units of DI-NH₂. Amine contaminants were then removed from DI-NH₂ though repeated desalting steps. The necessary volume of S-4FB was determined and reacted with DI-NH₂ for 2 hours at room temperature. Excess S-4FB was removed using centrifugal filtration (5 kDa MWCO). To determine the molar substitution ratio of 4FB modified DI (4FB-DI), 2 of 4FB-DI reacted with 18 μL of 2-HP solution and the UV-Vis absorbance spectra was measured. Untagged VCAM-1 monoclonal antibodies were then modified with amino-reactive S-HyNic. 100 μg of VCAM-1 antibodies were repeatedly desalted to remove amine contaminants. 2 μL S-HyNic (2.85 g/L) were added to the desalted antibody solution and allowed to react for 2 hours at room temperature. Excess S-HyNic was removed using centrifugal filtration (50 kDa MWCO). 4FB-DI and HyNic-antibodies were reacted for 2 hours in 1×Turbolink Catalyst Buffer. DI-antibody conjugates were verified through analysis of UV-Vis absorbance spectra. A characteristic peak (λ=354 nm) was observed as a result of HyNic-4FB linkage.

Bidirectional Fluorescent Labeling of Non-Adherent CCRF-CEM Cells

Characterization of the fluorescent amplification and reversal reactions using DM₂-Alginate-Biotin conjugates was assessed by flow cytometric experiments and fluorescence microscopy. Non-adherent CCRF-CEM cells were selected due to their high compatibility with flow cytometric analyses. Enzymes and chelating agents required for cell dissociation may contribute to the degradation of the DNA nanoscaffolds due to membrane damage and destabilization of DNA strand.

DNA nanostructures were generated on the surface of CCRF-CEM cells via the hybridization chain reaction (HCR) between FAM-labeled DM1 sequences (DM1-FAM) and purified DM2-Alginate-Biotin conjugates. CCRF-CEM cells were rinsed with DPBS and resuspended at 1×10⁶ cells/mL. Cholesterol-modified DNA initiators (DI-Cholesterol) were added to CCRF-CEM cells to a final concentration of 50 nM. Excess DI-Cholesterol was removed from DI-modified CCRF-CEM cells (DI-cells) by three DPBS rinsing steps. To generate Alginate-HCR labeled samples, DI-cells were suspended in a 1 μM solution of DM1-FAM and DM2-Alginate-Biotin for 3 hours at room temperature. To generate FAM labeled samples, DI-cells were suspended in a 1 μM solution of DM1-FAM for 3 hours at room temperature. To generate PE-Cy5.5 labeled samples, DI-cells were suspended in a 1 μM solution of biotinylated DI complementary sequence (CSDI-Biotin) for 3 hours at room temperature. To generate Alginate labeled samples, DI-cells were suspended in a 1 μM solution of DM1-FAM for 1.5 hours at room temperature, rinsed three times with DPBS, then suspended in a 1 μM solution of DM2-Alginate-Biotin for 1.5 hours at room temperature. All samples were rinsed with DPBS three times to remove excess oligonucleotides and resuspended in DPBS. 2 μL Streptavidin-PE-Cy5.5 (0.05 mg/mL) were added to resuspended samples for 30 minutes at room temperature.

Alginate-HCR samples were then reversed following the addition of complementary DNA trigger sequences (T_{DI}, T_{DM1}) and alginate lyase. A 10:1 ratio of complementary DNA triggers to expected displacement sites was calculated to determine the concentration of DNA trigger molecules. To generate T_{DI} samples, Alginate-HCR samples were suspended in a 0.5 μM solution of T_{DI} for 30 minutes at room temperature. To generate T_{DM1} samples, Alginate-HCR samples were suspended in a 2.5 μM solution of T_{DM1} for 30 minutes at room temperature. To generate T_{DI}+T_{DM1} samples, Alginate-HCR samples were suspended in a solution of T_{DI} (0.5 μM) and T_{DM1} (2.5 μM) for 30 minutes at room temperature. To generate T_{DI}+T_{DM1}+Alginase samples, Alginate-HCR samples were suspended in a solution of T_{DI} (0.5 μM), T_{DM1}(2.5 μM), and alginate lyase (1 unit) for 30 minutes at room temperature.

Mean fluorescent intensity was measured by flow cytometry analysis for all samples. Signal-to-noise ratio (SNR) was calculated for all labeled samples. Remaining fluorescent signal of triggered Alginate-HCR samples was calculated as a percentage of the initial Alginate-HCR intensity. Representative fluorescent images were captured for each labeled and triggered sample using an exposure time of 1 second and a lamp intensity of 25%.

Antibody-Mediated Bidirectional Fluorescent Labeling of Adherent C166 Cells

C166 endothelial cells were selected due to constitutive expression of a known biomarker, VCAM-1. The strong attachment of C166 cells also allows them to remain adherent throughout repetitive rinsing steps. C166 cells were seeded at a density of 20,000 cells/well in a 24-well plate. 5 μL of either FITC-labeled VCAM-1 antibodies (0.5 mg/mL) or DI-conjugated VCAM-1 antibodies (˜0.5 mg/mL) were added to 250 μL DPBS. Loosely bound antibodies were removed by rinsing each well with DPBS three times. FITC-antibody samples were imaged immediately to avoid photobleaching of fluorescent signal over time. Meanwhile, DNA nanostructures were generated on the surface of C166 cells. To generate Alginate-HCR labeled samples, 3004, of a solution of DM1-Cy5 (1 μM) and DM2-Alginate-AlexaFluor488 (1 μM) was added to the well for 3 hours at room temperature. All samples were rinsed with DPBS 3 times to remove excess oligonucleotides. The fluorescent signal of Alginate-HCR samples was then reversed by 300 μL of a solution containing T_{DI} (1 μM), T_{DM1}(1 μM) and alginate lyase (1 unit). Representative fluorescent images were captured for each labeled and triggered sample using an exposure time of 1 second and a lamp intensity of 25%.

Results

First, the ability of the DNA hairpin conjugates to hybridize into nanoscaffolds for fluorescent labelling applications was examined (FIG. 1). After the cells were labeled with a DI-cholesterol probe that is inserted into the cell membrane, the cells were contacted with two hairpin DNAs (DM1 and DM2) and hybridization chain reaction (HCR) is allowed to proceed. In this experiment, DM1 was labeled with FAM (as a control) and DM2 was provided as a DM2-Alginate-Biotin conjugate. Following HCR, the nanoscaffold is fluorescently labeled with PE-Cy5.5-steptravidin conjugates that bind to the biotin moiety on the DM2-Alginate-Biotin conjugate. As is depicted schematically in FIG. 1A, this alginate-HCR treatment (“Alginate-HCR”) was compared to several controls, including unlabeled cells (“unlabeled”) to show the baseline fluorescence of the cells; cells labeled with the DI-cholesterol probe and DM1-FAM (“FAM”) to show labeling with a single fluorophore; cells labeled with the DI-cholesterol probe and a complementary sequence conjugated to a single PE-Cy5.5-steptravidin unit (“PE-Cy5.5”) to show labeling with a single PE-Cy5.5-streptavidin conjugate; and cells labeled with the DI-cholesterol probe, DM1-FAM, and a single DM2-Alginate-Biotin conjugate (“Alginate”) to show labeling with a single HCR repeat unit (generated by one-step hybridization). No significant difference was observed between the mean fluorescent intensity readings of the FAM and single unit labeled groups (i.e., Alginate). However, the polyvalent DNA nanomaterials generated under HCR conditions exhibited FAM intensity values over 5 times greater than either the FAM or single unit labeled sample (FIG. 1B). Signal-to-noise ratios (SNR) were calculated using Equation 1:

$$SNR = \frac{{μ}_{sample}}{{σ}_{unlabeled}} \qquad ( 1 )$$

FAM SNR values demonstrate the linear nature of signal amplification produced by polymerization of the DNA backbone (FIG. 1C). Fluorescent imaging shows the localization and enhancement of FAM expression on the cell surface due to the repeat DM1-FAM sequences of the Alginate-HCR labeled sample (FIG. 1D).

Furthermore, mean PE-Cy5.5 intensity values show a nearly three times increase in signal intensity between the PE-Cy5.5 and single unit labeled samples. The single unit labeled PE-Cy5.5 signal is additionally enhanced by nearly 5-fold to achieve the HCR labeled intensity values (FIG. 1E). PE-Cy5.5 SNR values indicate a two-layer amplification mechanism. A comparison of the Alginate-HCR and single unit labeled groups demonstrates the significance of DNA polymerization to signal enhancement, while the contributions of alginate side-branching are exhibited through comparison of the single unit and PE-Cy5.5 labeled samples (FIG. 1F). The increased fluorescent expression of both the Alginate-HCR and the single unit groups is demonstrated in the PE-Cy5.5 channel fluorescent images (FIG. 1G).

The use of two fluorophores, FAM and PE-Cy5.5, allowed for the characterization of the hybridization efficiency via fluorescent intensity changes observed in flow cytometry experiments. The degree of DNA polymerization was determined by tracking the number of DM1-FAM repeat units present in each DNA chain, while the streptavidin-PE-Cy5.5 signal indicated the effective number of biotin sites present on the alginate branches. Consistency between the FAM and PECy5.5 signals confirmed the accuracy of the five repeat units achieved in the Alginate-HCR labeled samples. Qualitative analysis of fluorescent images demonstrated the critical importance of signal amplification techniques in the observation of live cells. Lastly, PE-Cy5.5 signals showcase the maximum amplification power by utilizing fluorescently-labeled streptavidin probes, HCR polymerization and biotinylated alginate branches.

Additionally, we demonstrated the ability to reverse the fluorescent signal utilizing only complementary DNA (cDNA) trigger sequences (T_{DI}, T_{DM1}; FIG. 2). T_{DI} is a cDNA that is complementary to the initiator DNA (DI), and T_{DM1} is a cDNA that is complementary to the first DNA hairpin (DM1). Variation in time, concentration, and triggering mechanisms affected the reduction of signal intensity (data not shown). Toehold-mediated strand-displacement (TMSD) was applied to the HCR labeled samples (FIG. 2A). In toehold-mediated strand displacement, a single-stranded overhand (or toehold) in DM1 promotes hybridization with the triggering cDNA. Once initially hybridized, the triggering cDNA displaces the original strand due to increased energetic favorability. Under reasonable conditions, a 3 hours incubation at 10:1 molar ratio and 25° C., FAM expression was reduced by all triggering mechanisms (FIG. 2C). A minimum FAM intensity reduction of more than 85% was observed, with the synergistic combination of T_{DI} and T_{DM1} resulting in a peak reversibility of over 95% of the HCR labeled FAM signal (FIG. 2D). Conversely, PE-Cy5.5 reversibility showed minimal signal reduction for all triggering sequences (FIG. 2F). PE-Cy5.5 intensity was reduced by 30%, 38%, and 57% after treatment with T_{DI}, T_{DM1}, and T_{DI}+T_{DM1}, respectively. (FIG. 2G).

Next, the ability of alginate lyase (i.e., alginase) to further reduce the fluorescent expression of the PECy5.5 stained alginate sidechains was investigated (FIG. 3). The alginase enzyme was added to the combination of T_{DI} and T_{DM1} and compared with the alginase-free sample. The addition of alginase had little impact on the reduction of FAM intensity values (FIG. 3A). Despite the statistical significance between the FAM SNR values of the alginase and alginase-free groups, the enzyme had little impact (˜1%) on the reversal of FAM signal intensity (FIG. 3B). Nevertheless, the incorporation of alginase encouraged the cleavage of alginate sidechains, leading to a dramatic reduction in the PE-Cy5.5 signal expression (FIG. 3D). Alginase improved the reduction of PE-Cy5.5 signal by over 1.5 times relative to the alginase-free sample. PE-Cy5.5 SNR was reduced from an HCR value of 185 to an alginase-enhanced reversibility value of 10.7 (FIG. 3E). Alginase-enhanced reversibility showed little additional benefit to the DNA triggering sequences in the captured FAM fluorescent images (FIG. 3C). In contrast, alginase significantly aided in the reversal of PE-Cy5.5 intensity, leading to a nearly 95% reduction in the HCR labeled signal. The magnitude of this improvement is clearly shown in the comparison of PE-Cy5.5 fluorescent images (FIG. 3F). The percentage of signal remaining in each condition tested in FIGS. 2 and 3 is reported in Table 2 (FAM) and Table 3 (PE-Cy5.5) below.

TABLE 2
Percentage of FAM signal remaining
relative to the Alginate-HCR group

| | FAM Signal |
|---|---|
| Condition | Remaining |
| Alginate-HCR | 100% |
| T_{DI} | 13.9% |
| T_{DM1} | 11.8% |
| T_{DNA} | 4.3% |
| T_{DNA} + Alginase | 3.3% |
| DNAse | 2.7% |

TABLE 3
Percentage of PE-Cy5.5 signal remaining
relative to the Alginate-HCR group

| | PE-Cy5.5 Signal |
|---|---|
| Condition | Remaining |
| Alginate-HCR | 100% |
| T_{DI} | 69.8% |
| T_{DM1} | 62.3% |
| T_{DNA} | 43.0% |
| T_{DNA} + Alginase | 5.8% |
| DNAse | 24.9% |

To test whether the bidirectional labeling system could be targeted to a specific biomolecule, C166 endothelial cells were selected due to their constitutive expression of the known biomarker VCAM-1 (FIG. 4). The cells were labeled with either FITC-labeled VCAM-1 antibodies (“FITC-Antibody”) or DI-conjugated VCAM-1 antibodies, and DNA nanostructures were generated on the surface of the cells labeled with DI-conjugated VCAM-1 antibodies (“HCR”) via incubation with DM1 and DM2-Alginate conjugates that were labeled directly with FITC fluorophores. The nanostructures were then dissociated using a combination of T_{DI}, T_{DM1}, and alginate lyase (“Triggered”). The results were consistent with those obtained using the CCRF-CEM model system: the HCR labeled cells showed increased signal intensity as compared to the FITC labeled cells (FIG. 4A-B), and the HCR signal was reversed by the treatment with T_{DI}, T_{DM1}, and alginate lyase (FIG. 4C).

### DISCUSSION

These antibody-mediated fluorescent labeling studies demonstrate that antibodies maintained their protein specificity after DNA initiator sequence conjugation, meaning that the conformational structure of the functional antigen-binding regions suffered no significant damage. Additionally, DNA hybridization was not hindered by the bulky conjugates utilized in the construction of DNA nanomaterials. Ultimately, these studies demonstrate that alginate-DNA conjugates retain their ability to efficiently self-hybridize into nanoscaffolds, avoiding potential complications such as steric hindrance. Fluorescent expression of CCRF-CEM lymphoblasts analyzed by flow cytometry and immunofluorescence imaging suggests the proposed method can enhance the specificity and detectability of protein biomarkers expressed on the cell surface through signal amplification. Moreover, DNA-alginate nanoscaffolds constructed via the HCR can be efficiently reversed for de-staining purposes. Thus, the incorporation of fluorescently labeled streptavidin probes and the unique destaining capabilities make this HCR-based signal amplification strategy worth further optimization and commercialization.

We claim:

1. A method for reversibly detecting a target analyte in a sample, the method comprising:
a) contacting the sample with a probe that comprises an initiator single-stranded DNA molecule (ssDNA) conjugated to a targeting agent that binds to the target analyte;
b) washing the sample to remove unbound probe;
c) contacting the probe-target analyte complex with:
i. a first DNA hairpin comprising (1) a first portion that is complementary to both a part of the initiator ssDNA and a part of a second DNA hairpin, and (2) a second portion that is complementary to a part of a first portion of the second DNA hairpin; and
ii. the second DNA hairpin comprising a first portion that is complementary to a part of the second portion of the first DNA hairpin and a second portion that is complementary to a part of the first portion of the first DNA hairpin;
wherein either the first hairpin or the second hairpin is linked to an alginate; and
wherein the initiator ssDNA, the first DNA hairpin, and the second DNA hairpin undergo hybridization chain reaction (HCR) when in contact, thereby forming a nanoscaffold attached to the targeting agent; and
d) contacting the nanoscaffold of step (c) with a detectable label that binds to the alginate; and
e) detecting the detectable label.

2. The method of claim 1, wherein the signal produced by the detectable label is increased multifold as compared to the signal produced in the absence of HCR.

3. The method of claim 2, wherein the signal produced by the detectable label is increased by at least 3-fold compared to the signal produced in the absence of HCR.

4. (canceled)

5. The method of claim 1, further comprising: (f) removing the detectable label from the sample by contacting the sample with a depolymerization agent selected from a complementary DNA (cDNA), alginate lyase, DNase, or any combination thereof.

6. The method of claim 5, wherein the depolymerization agent comprises a cDNA that is complementary to at least a portion of the initiator ssDNA, a cDNA that is complementary to at least a portion of the first DNA hairpin, a cDNA that is complementary to at least a portion of the second DNA hairpin, or any combination thereof.

7. The method of claim 5, wherein the depolymerization agent comprises a cDNA and an alginate lyase.

8. The method of claim 5, further comprising: (g) repeating steps (a)-(e) using a different targeting agent to detect a different target analyte.

9. The method of claim 1, wherein the targeting agent is an antibody or a nucleic acid aptamer that specifically binds to the target analyte.

10. The method of claim 1, wherein the alginate is conjugated to a binding agent and the detectable label is conjugated to a binding partner, and wherein the detectable label binds to the alginate via the interaction of the binding agent and the binding partner.

11. The method of claim 10, wherein the binding agent is biotin and the binding partner is streptavidin.

12. The method of claim 1, wherein the detectable label is directly linked to the alginate.

13. The method of claim 1, wherein the sample comprises one or more cells.

14. The method of claim 13, wherein the target analyte is a cell surface biomolecule.

15. The method of claim 13, wherein the wherein the target analyte is an intracellular biomolecule, and wherein the method further comprises prior to step (a): fixing and permeabilizing the cells in the sample.

16. The method of claim 13, wherein sample is a biopsy.

17. The method of claim 1, wherein the second DNA hairpin is linked to the alginate conjugated to a binding agent.

18. The method of claim 1, wherein the detectable label is a fluorophore.

19. The method of claim 18, wherein the fluorophore is selected from fluorescein, FAM (6-fluorescein amidite), sulforhodamine 101, pyrenebutanoate, acridine, ethenoadenosine, eosin, rhodamine, 5-(2′-aminoethyl)aminonaphthalene (EDANS), fluorescein isothiocyanate (FITC), N-hydroxysuccinimidyl-1-pyrenesulfonate (PYS), tetramethylrhodamine (TAMRA), Rhodamine X, Cy5, and erythrosine.

20. The method of claim 1, wherein the sample is washed to remove excess first DNA hairpin and second DNA hairpin before step (d).

21. A kit for detecting a target analyte in a sample, the kit comprising:
a) a probe that comprises an initiator single-stranded DNA molecule (ssDNA) conjugated to a targeting agent that binds to the target analyte;
b) a first DNA hairpin comprising (1) a first portion that is complementary to both a part of the initiator ssDNA and a part of a second DNA hairpin, and (2) a second portion that is complementary to a part of a first portion of the second DNA hairpin;
c) a second DNA hairpin comprising a first portion that is complementary to a part of the second portion of the first DNA hairpin and a second portion that is complementary to a part of the first portion of the first DNA hairpin; wherein the first DNA hairpin or second DNA hairpin is linked to alginate; and
d) a detectable label that binds to the alginate or is conjugated to the alginate.

22-26. (canceled)
